(19) United States
(12) Patent Application Publication
(10) Pub. No.: US 2026/0021072 A1
(43) Pub. Date: Jan. 22, 2026

(54) METHOD FOR WOUND TREATMENT

(71) Applicants: National Yang Ming Chiao Tung University, Hsinchu City (TW); Far Eastern Memorial Hospital, New Taipei City (TW)

(72) Inventors: Ting-Ting Chang, Taipei City (TW); Yen-Wen Wu, Taipei City (TW); Jaw-Wen Chen, Taipei City (TW)

(73) Assignees: National Yang Ming Chiao Tung University, Hsinchu City (TW); Far Eastern Memorial Hospital, New Taipei City (TW)

(21) Appl. No.: 19/235,636

(22) Filed: Jun. 12, 2025

(30) Foreign Application Priority Data
Jul. 17, 2024 (TW) 113126785

(51) Int. Cl. A61K 31/415; A61M 31/00; A61P 9/10; A61P 17/02; A61P 29/00

(52) U.S. Cl. CPC A61K 31/415; A61M 31/00; A61P 9/10; A61P 17/02; A61P 29/00

(57) ABSTRACT

A method for wound treatment includes administering a subject in need thereof with a pharmaceutical composition. The pharmaceutical composition includes: an effective amount of a fatty acid-binding protein 4 (FABP4) antagonist and/or a pharmaceutically acceptable salt thereof; and a pharmaceutically acceptable carrier. The wound treatment may include promoting wound angiogenesis, enhancing wound healing, reducing wound inflammation, and/or inhibiting wound scarring.

### CROSS REFERENCE TO RELATED APPLICATION

This application claims the benefit of TW application No. 113126785, filed on Jul. 17, 2024. The content of the application is incorporated herein by reference.

### BACKGROUND OF THE INVENTION

### 1. Field of the Invention

The present disclosure relates to a method for wound treatment, and more particularly to a method for wound treatment by administration of a fatty acid-binding protein 4 (FABP4) antagonist.

### 2. Description of the Prior Art

The skin protects the body by preventing harm and infection from external substances. The process of skin wound healing involves complex and interrelated biochemical reactions, including hemostasis, inflammation, proliferation, and remodeling, to repair tissue. To this day, skin wound care and how to promote wound healing remain important topics and challenges in clinical practice. Furthermore, persistent inflammation and infection can easily lead to scarring during the wound healing process, which is one of the problems to be solved in the art.

Although minor wounds in healthy subjects usually heal efficiently, various external and internal factors, such as infection, genetic defects, age, malnutrition, improper vascularization, and/or poor angiogenesis, may weaken or postpone the process of wound healing. Even when minor or acute wounds occur in the healthy subject without underlying disease, suboptimal wound healing may lead to undesirable consequences, such as wound scarring, wound inflammation, slow wound recovery, or increased susceptibility to complications.

However, there is still an unmet need in the art for an approach to wound treatment that can solve the aforementioned problems simultaneously.

### SUMMARY OF THE INVENTION

In view of the foregoing, the present disclosure provides a method for wound treatment. The method includes administering a subject in need thereof with a pharmaceutical composition, and the pharmaceutical composition includes: an effective amount of a fatty acid-binding protein 4 (FABP4) antagonist and/or a pharmaceutically acceptable salt thereof; and a pharmaceutically acceptable carrier.

In at least one embodiment of the present disclosure, the method for wound treatment effectively enhances wound healing, promotes wound angiogenesis, and/or reduces wound inflammation, thereby inhibiting wound scarring in not only chronic wounds but also acute wounds of subjects.

The objectives of the present disclosure will no doubt become understandable to those of ordinary skill in the art after reading the following detailed description of the embodiments that are illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

By reading the detailed description below and the figures described herein, the implementation modes of the present disclosure will be further understood.

FIG. 1A shows the distribution of serum FABP4 concentrations in mice from the normal diet group (ND), the untreated HFD group (−), the HFD group administered with 5 mg/kg/day BMS309403 (5), and the HFD group administered with 25 mg/kg/day BMS309403 (25). FIG. 1B and FIG. 1C show the levels of inflammatory markers, IL-6 and TNF-α, respectively that are decreased in the HFD groups administered with 5 mg/kg/day or 25 mg/kg/day BMS309403 compared with the untreated HFD group suffering from the HFD-induced metabolic dysfunction. *P<0.05 and #P<0.01.

FIG. 2A and FIG. 2B show the wound healing status of mice from the normal diet group (ND), the untreated high-fat diet group (HFD), the HFD group administered with 5 mg/kg/day BMS309403 (BMS 5), and the HFD group administered with 25 mg/kg/day BMS309403 (BMS 25). FIG. 2A presents photographs of the wounds at days 0, 1, 3, and 5 for each group of mice, and FIG. 2B displays a line graph showing the changes in wound healing at each time point for the different groups. Compared to the normal diet group (ND), *P<0.05 and ** P<0.01; compared to the untreated HFD group (HFD), #P<0.05.

FIG. 3A to FIG. 3E show representative wound area sections from the normal diet group (ND), the untreated HFD group (“HFD” in FIG. 3A, FIG. 3B. and FIG. 3D; “−” in FIG. 3C and FIG. 3E), the HFD group administered with 5 mg/kg/day BMS309403 (“HFD+BMS 5” in FIG. 3A, FIG. 3B, and FIG. 3D; “5” in FIG. 3C and FIG. 3E), and the HFD group administered with 25 mg/kg/day BMS309403 (“HFD+BMS 25” in FIG. 3A, FIG. 3B, and FIG. 3D; “25” in FIG. 3C and FIG. 3E). FIG. 3A shows the sections stained with H&E, with arrows indicating the wound position and size (the width between the two arrows). FIG. 3B displays representative wound area sections from each group of mice stained with anti-CD31 antibody, where CD31 serves as a biomarker for capillaries and angiogenesis. FIG. 3C shows the quantitative analysis of the CD31 antibody immunostaining. FIG. 3D shows the expression of FABP4 in the wound area. FIG. 3E shows the quantitative analysis of FABP4 antibody immunostaining. * P<0.05 and #P<0.01.

FIG. 4A and FIG. 4B show images of the western blot analysis and the quantification result thereof, respectively, illustrating the level of angiogenic factors, p-Akt, Akt, p-eNOS, eNOS, SDF-1, and VEGF, in the wound area of the normal diet group (ND), the untreated HFD group (−), the HFD group administered with 5 mg/kg/day BMS309403 (5), and the HFD group administered with 25 mg/kg/day BMS309403 (25). p-Akt/Akt and p-eNOS/eNOS shown in FIG. 4B indicate the normalized degree of Akt phosphorylation and eNOS phosphorylation, respectively. Actin, a housekeeping protein, is used as a loading control for the western blot analysis. The Action expression in lanes 6 and 8 of FIG. 4A is used to normalize the expression of SDF-1 and VEGF, respectively, and the normalized results thereof are presented in SDF-1/Actin and VEGF/Actin of FIG. 4B. * P<0.05 and #P<0.01.

FIG. 5A and FIG. 5B shows images of the western blot analysis and the quantification results thereof, respectively, of inflammatory factors, IL-1β, IL-6, and TNF-α, in the wound area of the normal diet group (ND), the untreated HFD group (HFD), the HFD group administered with 5 mg/kg/day BMS309403 (5), and the HFD group administered with 25 mg/kg/day BMS309403 (25). * P<0.05 and #P<0.01.

FIG. 6 shows the quantification of the wound area of human aortic endothelial cells in wound healing assay in vitro. The wound areas of the untreated group (Control), the group administered with 5 μM BMS309403, and the group administered with 20 μM BMS309403 are recorded at 0 h, 6 h, and 12 h after administration. Quantification of wound area of human aortic endothelial cells from the control group and FABP4 inhibitor (BMS309403) treated groups at 0, 6, and 12 hours. * P<0.05 and #P<0.01.

### DETAILED DESCRIPTION

A person ordinarily skilled in the art will readily observe that numerous modifications and alterations of the present disclosure may be made while retaining the teachings of the disclosure described herein. Accordingly, the embodiments described below are intended to cover the modifications and alterations within the scope of the present disclosure, rather than to limit the present disclosure. The scope of the claims therefore should be accorded the broadest interpretation so as to encompass all such modifications and alterations.

The practice of the present disclosure employs, unless otherwise indicated, conventional techniques of molecular biology, microbiology, cell biology, biochemistry, and immunology, which are well within the purview of a skilled artisan in the art. Such techniques are explained fully in the literature, such as “Molecular Cloning shows A Laboratory Manual,” second edition (Sambrook, et al., 1989), Cold Spring Harbor Press; “Oligonucleotide Synthesis” (M. J. Gait, 1984); “Methods in Molecular Biology,” Humana Press; “Cell Biology shows A Laboratory Notebook” (J. E. Cellis, ed., 1998) Academic Press; “Animal Cell Culture” (R. I. Freshney, ed., 1987); “Handbook of Experimental Immunology” (Weir, 1996); “Introduction to Cell and Tissue Culture” (J. P. Mather and P. E. Roberts, 1998); “Cell and Tissue Culture shows Laboratory Procedures” (A. Doyle, J. B. Griffiths, and D. G. Newell, eds., 1993-8); “Methods in Enzymology” (Academic Press, Inc.); “Handbook of Experimental Immunology” (D. M. Weir and C. C. Blackwell, eds.); “Gene Transfer Vectors for Mammalian Cells” (J. M. Miller and M. P. Calos, eds., 1987); “Current Protocols in Molecular Biology” (F. M. Ausubel, et al., eds., 1987); “PCR shows The Polymerase Chain Reaction (Mullis, et al., eds., 1994); and “Current Protocols in Immunology” (J. E. Coligan et al., eds., 1991); “Short Protocols in Molecular Biology” (Wiley and Sons, 1999); “Immunobiology” (C. A. Janeway and P. Travers, 1997); “Antibodies” (P. Finch, 1997); “Antibodies shows a practical approach” (D. Catty., ed., IRL Press, 1988-1989); “Monoclonal antibodies shows a practical approach” (P. Shepherd and C. Dean, eds., Oxford University Press, 2000); “Using antibodies shows a laboratory manual” (E. Harlow and D. Lane (Cold Spring Harbor Laboratory Press, 1999); The Antibodies (M. Zanetti and J. D. Capra, eds., Harwood Academic Publishers, 1995). Particularly useful techniques for particular embodiments will be discussed in the sections that follow. Without further elaboration, it is believed that one skilled in the art can, based on the above descriptions, utilize the present disclosure to its fullest extent. The following embodiments are, therefore, to be construed as merely illustration, and not limitation of the remainder of the disclosure in any way whatsoever. All publications cited herein are incorporated by reference for the purposes or subject matter referenced herein.

As used herein, the singular forms “a,” “an,” and “the” are intended to include plural forms, unless the context clearly indicates otherwise. The terms “or” and “and/or” can be used interchangeably, unless it is otherwise indicated explicitly in the context.

As used herein, the terms “comprise,” “include,” “contain,” and any other variations thereof are intended to be inclusive in a manner of not excluding others. For example, when describing an object “comprises” a limitation, unless otherwise specified, it may additionally include other ingredients, elements, components, parts, steps, connections, etc., and should not exclude other limitations.

The terms “subject,” “individual,” and “patient” are used interchangeably in the present disclosure, and the term “subject” may be a human or anon-human animal. Examples of the subject include, but not limited to, human, monkey, mouse, rat, marmot, ferret, rabbit, hamster, cattle, horse, pig, deer, dog, cat, fox, wolf, chicken, emu, ostrich, and fish. In certain embodiments of the present disclosure, the subject is a mammal, e.g., a primate, such as a human.

The term “treat,” “treating,” or “treatment” is intended to encompass the alleviation or elimination of a condition, disease, or disorder, or one or more symptoms associated with that condition, disease, or disorder; or the mitigation or eradication of the underlying cause of the condition, disease, or disorder itself.

The term “prevent,” “preventing,” or “prevention” is intended to include methods for delaying and/or stopping the onset of a condition, disease, or disorder and/or its associated symptoms; preventing a subject from developing a condition, disease, or disorder; or reducing the risk of a subject suffering from a condition, disease, or disorder.

The term “improve,” “improving,” or “improvement” refers to the prevention or reduction of the severity or frequency of one or more symptoms or abnormalities exhibited by individuals with wounds, to any degree. Such improvement can be observed by the individuals receiving treatment or by others.

The term “effective amount” refers to the quantity of an active ingredient (such as FABP4 antagonists) administered to a subject that is sufficient to prevent or treat the development of a condition, disease, or disorder, or to alleviate that condition, disease, or disorder to some extent. As known to those skilled in the art, the effective amount can vary depending on the route of administration, the excipients used, other co-administered components, and the condition being addressed.

The numerical ranges used in this document are inclusive and combinable, and any value falling within these ranges can serve as either a maximum or minimum to derive subsequent sub-ranges. For example, the range “1 mg/kg to 100 mg/kg” should be understood to include any sub-range between the minimum value of 1 mg/kg and the maximum value of 100 mg/kg, such as from 1 mg/kg to 80 mg/kg, from 5 mg/kg to 70 mg/kg, or from 10 mg/kg to 60 mg/kg. Additionally, multiple values mentioned in this document may be selectively chosen as the highest and lowest values for derived numerical ranges. For instance, the values 1 mg/kg, 5 mg/kg, and 30 mg/kg can yield the ranges of 1 mg/kg to 5 mg/kg, 1 mg/kg to 30 mg/kg, and 5 mg/kg to 30 mg/kg.

The term “about” refers to an acceptable standard error of the mean considered by those skilled in the relevant field, which partly depends on how the value is measured or determined. In some embodiments, the term “about” refers to a range within 20%, 15%, 10%, 9%, 8%, 7%, 6%, 5%, 4%, 3%, 2%, 1%, 0.5%, or 0.05% of a given value or range. Unless otherwise expressly specified, all of the numerical ranges, amounts, values, and percentages such as those for quantities of materials, durations of time periods, temperatures, operating conditions, ratios of amounts, and the like disclosed herein should be understood as modified in all instances by the terms “about.”

This disclosure provides methods and uses for treating wounds and inflammation using pharmaceutical compositions, including administering an effective amount of fatty acid-binding protein 4 (FABP4) antagonists to inhibit FABP4 activity in subjects in need.

The term “administer,” “administering,” or “administration” refers to the placement of an active ingredient into a subject by a method or route that results in at least partial localization of the active ingredient at a desired site to produce the desired effect. The active ingredients described herein can be administered by any appropriate route known in the field, including but not limited to oral or parenteral routes, such as oral, sublingual, parenteral, rectal, intraperitoneal, intravenous, intradermal, inhalational, intramuscular, subcutaneous, intrapleural, topical, intranasal, or transdermal routes. In some embodiments of this disclosure, the pharmaceutical composition is administered orally to the subject.

In at least one embodiment of this disclosure, the pharmaceutical composition contains a fatty acid-binding protein 4 (FABP4) antagonist or a pharmaceutically acceptable salt thereof. In another embodiment of this disclosure, the pharmaceutical composition further includes a pharmaceutically acceptable carrier.

The term “pharmaceutically acceptable carrier” refers to a pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent, acid, surfactant, complexation agent, wetting agent, or any combination thereof. In some embodiments, each component is “pharmaceutically acceptable” in the sense of being compatible with the other ingredients of a pharmaceutical formulation, and suitable for use in contact with the organ or tissue of a subject (e.g., a mammal) without excessive toxicity, allergic response, irritation, immunogenicity, or other complications or problems. See, e.g., Remington: The Science and Practice of Pharmacy, 22nd ed.; Allen Ed.: Philadelphia, PA, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

FABP4, also known as fatty acid-binding protein 4, is a cytosolic fatty acid chaperone expressed in adipocytes and macrophages. The term “FABP4 antagonist” used herein includes any substance that, when administered to a subject, leads to the inhibition or downregulation of biological activity related to FABP4 in that individual, including the inhibition or downregulation of any downstream biological effects resulting from the binding of FABP4 to its receptor. FABP4 antagonists include any agents that can inhibit FABP4 activity or block the activation of FABP4 receptors or any downstream biological effects resulting from FABP4 receptor activation. Such FABP4 antagonists include any agents capable of interacting with FABP4 to prevent or reduce its normal biological activity. For example, the agents may be small molecules or antibodies targeting FABP4, such as FABP4 neutralizing antibodies that can block the interaction between FABP4 and its receptor or inhibit FABP4 activity. FABP4 antagonists may also include small molecules or antibodies targeting FABP4 receptors, which can exert their effects by occupying the ligand-binding site or a portion thereof on the receptor, thereby preventing the receptor from approaching FABP4.

At least one embodiment of the present disclosure provides a method for wound treatment, comprising administering a subject in need thereof with a pharmaceutical composition, wherein the pharmaceutical composition includes an effective amount of a fatty acid-binding protein 4 (FABP4) antagonist and/or a pharmaceutically acceptable salt thereof, along with a pharmaceutically acceptable carrier. At least one embodiment of the present disclosure provides a use of a fatty acid-binding protein 4 (FABP4) antagonist in the manufacture of a pharmaceutical composition for treating a wound in a subject in need thereof, wherein the pharmaceutical composition includes an effective amount of the FABP4 antagonist and/or a pharmaceutically acceptable salt thereof, along with a pharmaceutically acceptable carrier.

In at least one embodiment of the present disclosure, the FABP4 antagonist may be at least one selected from the group consisting of FABP4 small molecule antagonists, FABP4 neutralizing antibodies, and FABP4 gene interference ribonucleic acids.

In at least one embodiment of the present disclosure, the FABP4 antagonist may be BMS309403 or the pharmaceutically acceptable salt thereof.

In at least one embodiment of the present disclosure, the effective amount of the FABP4 antagonist may be about 1 mg/kg to about 100 mg/kg, such as but not limited to about 5 mg/kg to about 25 mg/kg. In some embodiments, the effective amounts of the FABP4 antagonist may be about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg, but the present disclosure is not limited thereto.

In at least one embodiment of the present disclosure, the administration duration of the FABP4 antagonist or the pharmaceutical composition is about 10 days. In another embodiment, the FABP4 antagonist or the pharmaceutical composition is continuously administered until wound healing occurs. In some embodiments, the administration duration of the FABP4 antagonist or the pharmaceutical composition may be about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, but the present disclosure is not limited thereto.

In at least one embodiment of the present disclosure, the frequency of administration of the FABP4 antagonist or the pharmaceutical composition may be three times a day, twice a day, once a day, once every two days, once every three days, once every four days, once every five days, once every six days, or once every seven days. In some embodiments, the frequency of administration of the FABP4 antagonist or the pharmaceutical composition may be once a week, twice a week, three times a week, four times a week, five times a week, six times a week, or seven times a week, but the present disclosure is not limited thereto.

In at least one embodiment of the present disclosure, the FABP4 antagonist may be administered before the wound occurs. In another embodiment, the FABP4 antagonist may be administered after the wound occurs.

In at least one embodiment of the present disclosure, the FABP4 antagonist and/or the pharmaceutically acceptable salt thereof may be the sole active ingredient for the wound treatment of the subject.

In at least one embodiment of the present disclosure, the wound may be from the subject suffering from metabolic syndrome and/or diabetes.

In at least one embodiment of the present disclosure, the pharmaceutical composition is used for promoting wound angiogenesis, enhancing wound healing, reducing wound inflammation, and/or inhibiting wound scarring. In some embodiments, the pharmaceutical composition may promote wound angiogenesis, enhance wound healing, reduce wound inflammation, and/or inhibit wound scarring in not only the chronic wound but also the acute wound.

The term “wound” used herein means any damage, injury, lesion, or other disruption of the normal structure or integrity of living tissue of a subject. The wound may include any break or discontinuity in tissue or any impairment of tissue function caused by trauma, disease, surgery, or other insult. In some embodiments, the wound may involve a loss of anatomical or functional integrity of the skin, underlying soft tissues, or organ surfaces. The term “wound” used herein may be acute wound or chronic wound, and the term is intended in its broadest reasonable sense to cover any such injury.

As used herein, the term “chronic wound” refers to a wound that fails to progress through the normal stages of healing in a timely and orderly manner and remains open, unhealed, or only partially healed for an extended period of time. In some embodiments, the extended period of time may be greater than three or four weeks. Chronic wounds may result from an underlying pathology that impairs the wound healing cascade, such as sustained inflammation, poor vascularization, infection, metabolic syndrome, diabetes, or systemic disease. Non-limiting examples of chronic wounds include diabetic ulcers, pressure ulcers, decubitus ulcers, venous stasis ulcers, arterial ulcers, vasculitic ulcers, trauma-induced ulcers, and ulcers associated with immunosuppression or ischemia. In some embodiments, the chronic wound may be an arterial ulcer that includes ulcerations resulting from complete or partial arterial blockage, or the chronic wound may be a venous stasis ulcer that includes ulcerations resulting from a malfunction of the venous valve and the associated vascular disease. The chronic wounds may be associated with comorbid conditions such as diabetes, obesity, peripheral vascular disease, or autoimmune disorders, which impair tissue repair mechanisms. In some embodiments, a chronic wound is a wound that exhibits delayed epithelialization, excessive inflammation, repeated infection, or recurrence after partial closure. The present disclosure contemplates treating chronic wounds of any type, cause, anatomical location, or severity.

As used herein, the term “acute wound” refers to a wound that typically progresses through the recognized phases of wound healing, i.e., hemostasis, inflammation, proliferation, and remodeling, within an expected timeframe. In some embodiments, the acute wound may be expected to heal within about three to four weeks, barring secondary complications. In some embodiments, the acute wounds may arise from surgical incisions, traumatic injuries (e.g., lacerations, abrasions, punctures), or burns, and are characterized by a predictable healing trajectory. The present invention contemplates enhancing or supporting the healing of acute wounds, including but not limited to those caused by surgery, accidental trauma, thermal injury, or cosmetic or reconstructive procedures. In some embodiments, the disclosed methods may be used to accelerate closure, reduce scar formation, or improve tissue regeneration in acute wounds.

In at least one embodiment of the present disclosure, the wound may be a site of a severe injury such as a traumatic acute, burn, chronic, or surgical wound for which the site has a compromised and/or missing outer layer of skin. Examples of wounds may include both closed and open wounds. Wounds may include, but not limited to, incisions, burns, excisions, ulcers, abrasions, puncture or penetrating wounds, lacerations, surgical wounds, contusions, hematomas, or crushing injuries. In some embodiments, the wound may include burns, venous stasis ulcers, pressure ulcers, delayed wound healing observed during corticosteroid treatment, delayed wound healing observed in the elderly (age-related defects), delayed wound healing observed in metabolic syndrome and/or diabetic patients, and epithelial defects following surgical scars or skin grafts. In some embodiments of this disclosure, the wound is that of a subject suffering from metabolic syndrome and/or diabetes. Diabetes and metabolic syndrome such as obesity are major risks for impaired wound healing that make clinical wound treatment more complicated and difficult. These diseases or disorders usually lead to chronic wounds and are often driven by high-fat dietary habits which may result in systemic metabolic disturbances, such as chronic inflammation, dyslipidemia, and/or insulin resistance. These pathophysiological changes disturb the normal wound healing cascade at multiple levels. When patients suffer from at least one of these high-fat diet-related disorders or diseases, the incidence of adverse clinical outcomes such as wound inflammation, delayed tissue repair, chronic wounds, and even systemic infection may be increased significantly.

In certain embodiments, the term “wound” includes injuries classified according to depth or severity. For example, wounds may be categorized as: (i) Grade I, limited to the epidermis; (ii) Grade II, extending into the dermis; (iii) Grade III, penetrating into the subcutaneous tissue; and (iv) Grade IV, or full-thickness wounds, which may expose bone or underlying structures such as the sacrum or greater trochanter. As used herein, the term “partial thickness wound” refers to wounds encompassing Grades I-III, including but not limited to superficial burns, pressure sores, venous ulcers, and diabetic ulcers. The term “deep wound” includes Grade III and Grade IV wounds. Unless specifically stated otherwise, all such wounds, regardless of type, classification, cause, chronicity, or anatomical location, are encompassed within the term “wound” as used in the present disclosure. The methods of the present invention contemplate treating all wound types, including deep wounds and chronic wounds.

As used herein, the term “wound healing” refers to the biological and physiological process involved in the repair and restoration of tissue following injury. Wound healing may include a series of coordinated phases including, but not limited to: hemostasis (e.g., platelet activation and clot formation), inflammation (e.g., immune cell recruitment), proliferation (e.g., epithelialization, fibroblast activation, angiogenesis), and remodeling (e.g., extracellular matrix reorganization and tissue maturation). As used herein, “enhancing wound healing” may include any therapeutic effect that facilitates or accelerates one or more of these processes. In some embodiments, enhancing wound healing may include but not limited to, reducing inflammation, promoting epithelial cell proliferation, increasing vascularization, stimulating tissue regeneration, or restoring healing capacity in wounds, regardless of whether the wound is acute or chronic in nature.

The term “angiogenesis” refers to the growth or formation of blood vessels.

Angiogenesis includes the growth of new blood vessels from pre-existing ones, as well as vasculogenesis (the spontaneous formation of blood vessels) and intussusception (the formation of new blood vessels by splitting existing ones). Angiogenesis encompasses “neovascularization,” “vascular regeneration,” “new vessel formation,” and “vascular remodeling.” Angiogenesis plays an important role in wound healing, especially in the proliferation phase. Poor angiogenesis in the wound region may lead to nutrient deficiency, waste accumulation, and tissue hypoxia, and the immune cells such as macrophages or reparative cells such as fibroblasts may fail to reach the wound region effectively. Accordingly, impaired angiogenesis may cause stalled wound healing, persistent inflammation, and increased susceptibility to infection of the wounds. These problems are particularly pronounced in patients suffering from metabolic diseases or disorders, where vascular dysfunction, impaired endothelial cell function, and chronic inflammation contribute to defective angiogenesis.

In at least one embodiment of the present disclosure, promoting wound angiogenesis includes increasing the expression of a wound angiogenic factor.

In at least one embodiment of the present disclosure, the angiogenic factor includes VEGF, SDF-1, EGF, Ang, EPO, FGF, GDF, eNOS, AKT, or combinations thereof. In another embodiment, the angiogenic factor may be at least one selected from the group consisting of VEGF, SDF-1, EGF, Ang, EPO, FGF, GDF, eNOS, AKT, and combinations thereof, with VEGF and AKT being preferred. In some embodiments, VEGF may be any member of the VEGF family, such as VEGF-A, VEGF-C, VEGF-D, or placental growth factor (PIGF). In some embodiments, AKT may be any member of the AKT family, such as AKT1, AKT2, and AKT3, and the mentioned AKT may be phosphorylated AKT (p-AKT).

In at least one embodiment of the present disclosure, reducing wound inflammation includes decreasing the expression of a wound inflammatory factor.

In at least one embodiment of the present disclosure, the inflammatory factor includes G-CSF, IL-1β, IL-1α, IL-2, IL-6, IL-8, IL-11, IL-17, IL-18, IFN-α, IFN-β, IFN-γ, TNF-α, TNF-β, or combinations thereof. In another embodiment, the inflammatory factor may be at least one selected from the group consisting of G-CSF, IL-1β, IL-1α, IL-2, IL-6, IL-8, IL-11, IL-17, IL-18, IFN-α, IFN-β, IFN-γ, TNF-α, TNF-β, and combinations thereof, with IL-1ß and TNF-α being preferred.

### EXAMPLES

Exemplary embodiments of the present disclosure are further described in the following examples, which should not be construed to limit the scope of the present disclosure.

### Materials and Methods

### Animals

Five-week-old male C57BL/6J mice were purchased from the National Experimental Animal Center (Taipei, Taiwan). After a two-week acclimatization period, some mice were fed a high-fat diet (HFD) for 12 weeks. In this high-fat diet, fat, carbohydrates, and protein provided 60.9%, 20.1%, and 18.3% of the calories, respectively. Mice fed a normal diet (ND) were used as a control group. Mice were randomly assigned to each group, with six mice per group. Starting 10 days prior to sacrifice (i.e., 3 days before wound creation), mice in the BMS309403 group receiving the high-fat diet were orally administered the FABP4 antagonist (BMS309403; 5 or 25 mg/kg/day; Cayman Chemical, Ann Arbor, MI, USA) for 10 days. Blood glucose levels were assessed after a 6-hour fast. All mice were anesthetized with isoflurane and sacrificed via cardiac puncture. Wound and dermal white adipose tissue (dWAT; a unique layer of adipocytes in the reticular dermis of the skin, functioning in antibacterial defense, wound healing, and thermogenesis) were collected. Following the regulations of the Animal Care Committee of National Yang Ming Chiao Tung University (Taipei, Taiwan), mice were maintained in a specific pathogen-free environment, housed in microisolator cages on a 12-hour light/dark cycle. All animal-related research was conducted in accordance with approved methods by the Animal Care Committee (IACUC) of National Yang Ming Chiao Tung University.

In vivo wound healing assessment and evaluation of wound morphology changes.

Full-thickness circular wounds with a diameter of 3 mm were created using a biopsy punch. Wound images were recorded and measured on days 0, 1, 3, and 5 using a digital camera (Nikon, Tokyo, Japan). Mice were sacrificed on day 7, and the wound tissue transverse sections were fixed in 4% paraformaldehyde, embedded in paraffin, and affixed to slides for staining. The tissues were sectioned into 5 μm thick samples and stained with hematoxylin and eosin (H&E) to evaluate changes in wound morphology.

### Capillary Density of Wounds

Sections were de-paraffinized and incubated with a rabbit-monoclonal antibody FABP4 (Abcam, ab92501; Cambridge, United Kingdom) and a rabbit-polyclonal antibody CD31 (Abcam, ab124432; Cambridge, United Kingdom). Antibody distribution was visualized with the avidin-biotin-complex technique and Vector Red chromogenic substrate, followed by counterstaining with hematoxylin. Sections were allowed to dry overnight for histological analysis.

### Assessment of FABP4 Levels in Mouse Serum

FABP4 concentrations were measured using ELISA according to the manufacturer's instructions (ELM-FABP4, Raybiotech Life Inc., Peachtree Corners, GA, USA).

### Western Blot Analysis

Samples were electrophoresed in SDS-polyacrylamide gels (10 to 12%), and proteins were transferred to polyvinylidene difluoride (PVDF) membranes. Membrane proteins were detected using monoclonal antibodies specific to AKT (BD Biosciences, 610868; NJ, USA), p-AKT (BD Biosciences, 550747; NJ, USA), endothelial nitric oxide synthase (eNOS; Cell Signaling, 32027S; Boston, MA, USA), p-eNOS (Santa Cruz Biotechnology, sc-81510; Dallas, TX, USA), vascular endothelial growth factor (VEGF; Santa Cruz Biotechnology, sc-7269; Dallas, TX, USA), stromal cell-derived factor 1 (SDF-1; Cell Signaling, 3740S; Boston, MA, USA), IL-1β (Santa Cruz Biotechnology, sc-52012; Dallas, TX, USA), IL-6 (Santa Cruz Biotechnology, sc-32296; Dallas, TX, USA), TNF-α (Santa Cruz Biotechnology, sc-52746; Dallas, TX, USA), and actin (Merck, MAB1501, Darmstadt, Germany). Bands were detected using enhanced chemiluminescence reagents.

### In Vitro Wound Healing Assessment

Human dermal microvascular endothelial cells (HDMECs; ScienCell, 2000; Carlsbad, CA, USA) were cultured in fibronectin (Corning, 356008; Tewksbury, MA, USA)-coated 6 well plates with endothelial cell medium (ECM; ScienCell, 1001; Carlsbad, CA, USA) containing 5% fetal bovine serum, 1% of endothelial cell growth supplement, and 1% penicillin/streptomycin solution at 37° C. in a humidified atmosphere of 5% CO2. Once the cell monolayer was confluent, a wound was created by scratching a straight line using a 200 μL pipette tip. Remove the debris by washing the cells once with 1 ml of the growth medium and then replace with 1 ml of growth medium containing 5% fetal bovine serum. Images were captured at 0, 6, and 12 hours post-scratch. Wound areas were quantified at each time point using ImageJ.

### Statistical Analysis

Results are expressed as mean±standard deviation. Statistical analyses were performed using unpaired Student's t-test or ANOVA followed by Scheffe's post hoc test. A p-value of <0.05 was considered statistically significant.

### Example 1: Reduction of Systemic Inflammation by FABP4 Inhibition

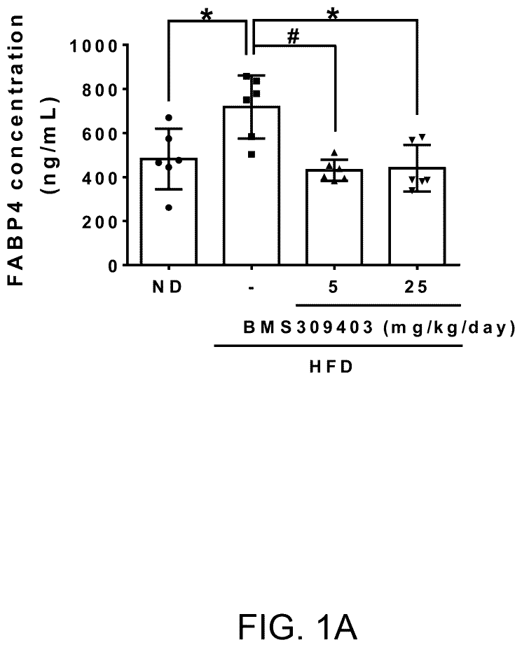

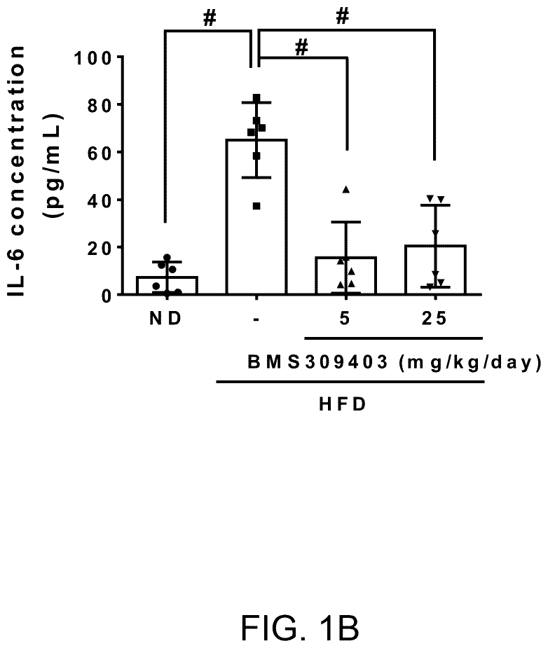

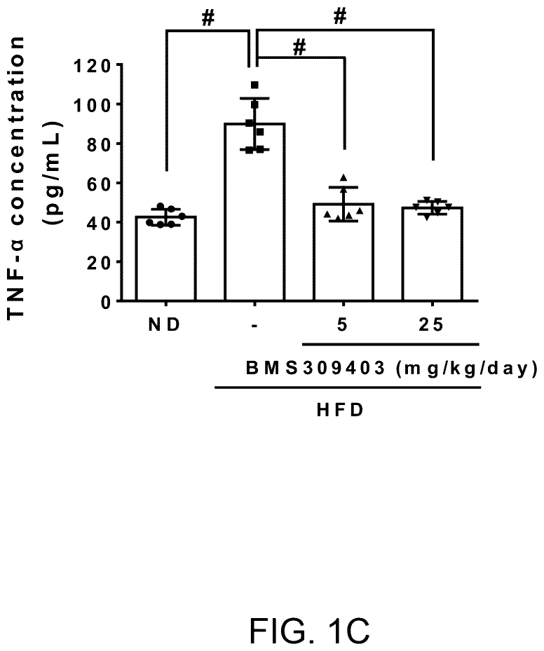

As shown in FIG. 1A, serum levels of FABP4 were significantly higher in mice fed a high-fat diet (HFD) compared to those fed a normal diet (ND). In mice with metabolic disorders induced by HFD, administration of the FABP4 antagonist, BMS309403, significantly reduced serum FABP4 levels. FIG. 1B and FIG. 1C further illustrate that levels of inflammatory markers, IL-6 and TNF-α, were significantly higher in the HFD mice compared to the ND mice, and administration of BMS309403 significantly reduced IL-6 levels and TNF-α levels in the HFD mice.

Therefore, these results indicate that the administration of BMS309403 can reduce the serum FABP4 levels of the mice with metabolic diseases or disorders induced by HFD, and the inhibition of FABP4 reduces systemic inflammation in the mice.

### Example 2: Enhancement of Wound Healing by FABP4 Inhibition

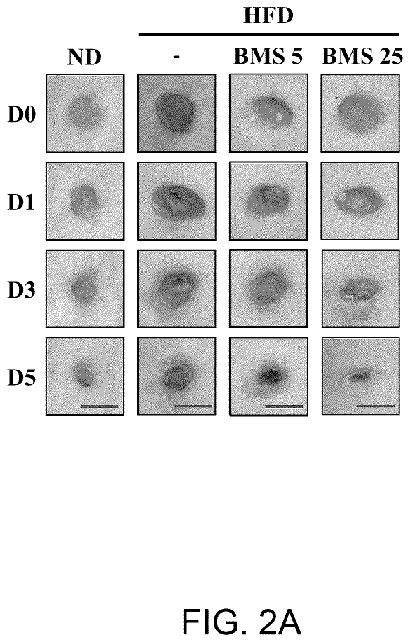

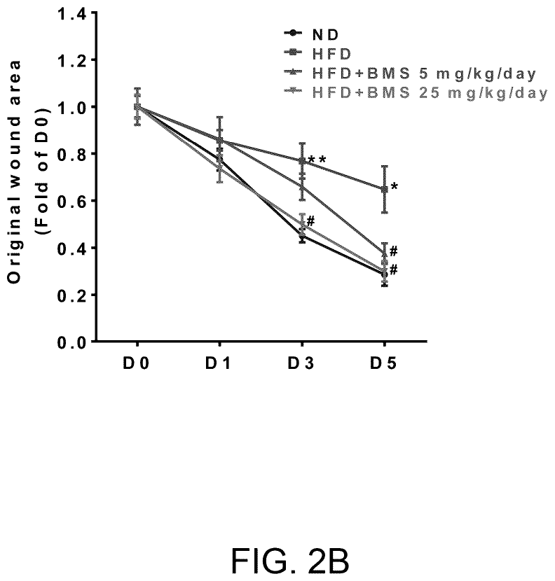

As shown in FIG. 2A, wound healing was significantly poorer in mice with metabolic diseases or disorders induced by high-fat feeding (HFD) compared to mice fed a normal diet (ND), illustrating the animal model of chronic wounds. In contrast, the HFD mice treated with BMS309403 exhibited faster wound healing compared to the untreated HFD mice. Quantification (FIG. 2B) revealed that on day 3 post-wounding, wound healing was significantly slower in the HFD mice compared to the ND mice. However, compared to untreated HFD mice, BMS309403-treated HFD mice showed accelerated healing on days 3 and 5 post-wounding.

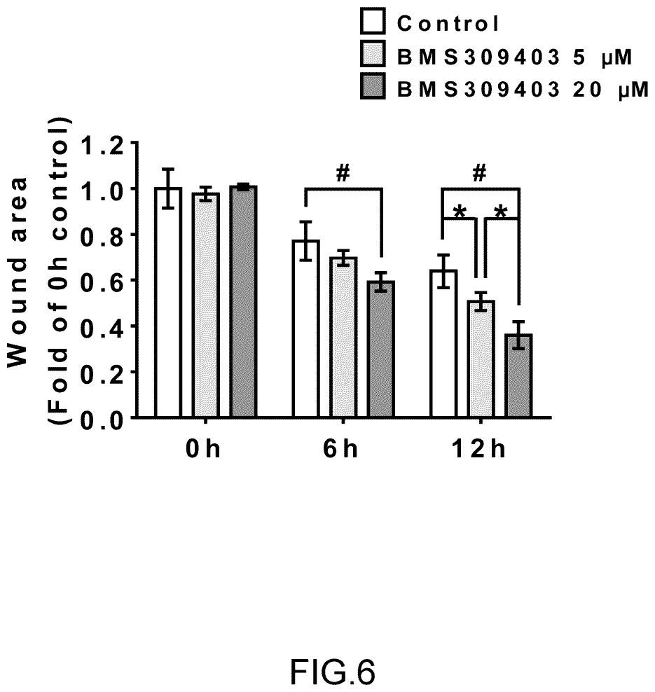

As shown in FIG. 6, the in vitro wound healing assay using human aortic endothelial cells (HAECs) demonstrated that BMS309403 significantly promotes cell migration and proliferation, thereby enhancing healing of the wound area at 6 h and 12 h post-wounding. These biological processes are central to the acute wound healing response, particularly during the early stages of re-endothelialization following injury. Further, the in vivo wound healing assay of ND mice demonstrated that the BMS309403-treated ND mice had faster wound healing than untreated ND mice (data not shown).

Therefore, these results indicate that inhibition of FABP4 enhances wound healing for not only chronic wounds but also acute wounds.

### Example 3: Promotion of Angiogenesis at the Healed Wound Site by FABP4 Inhibition

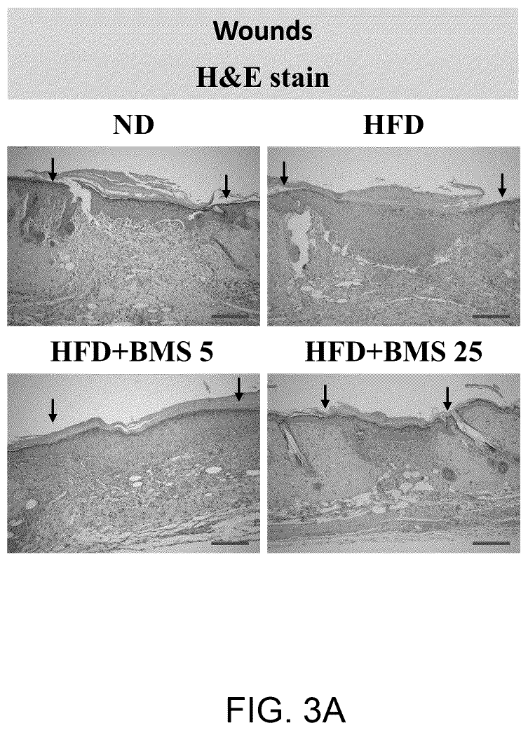

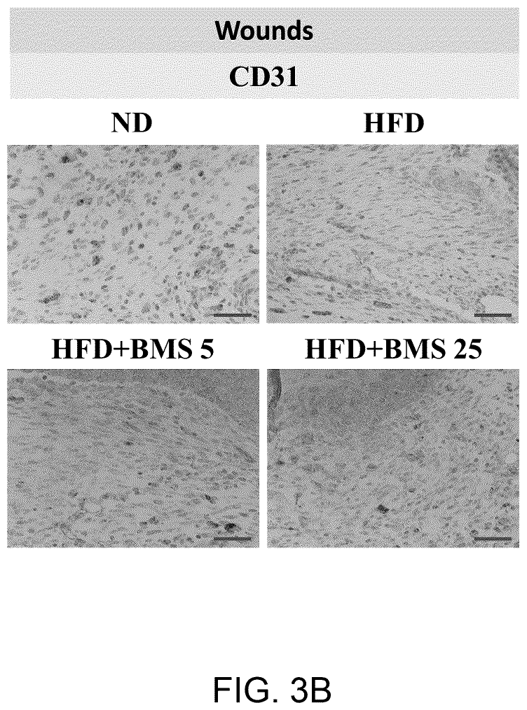

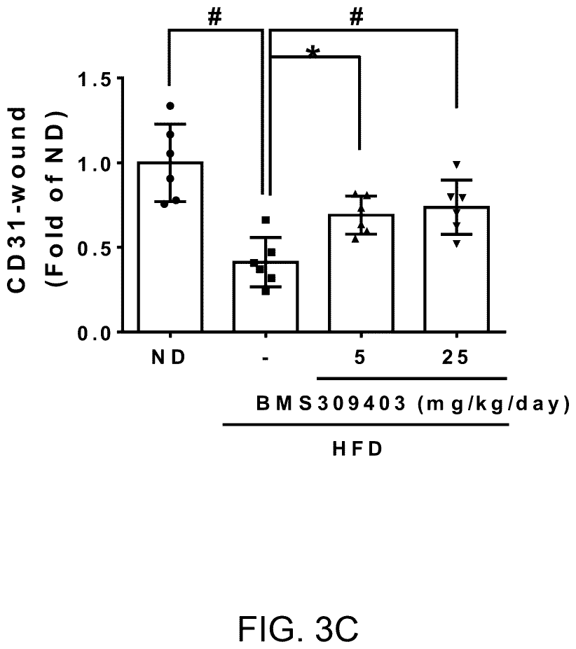

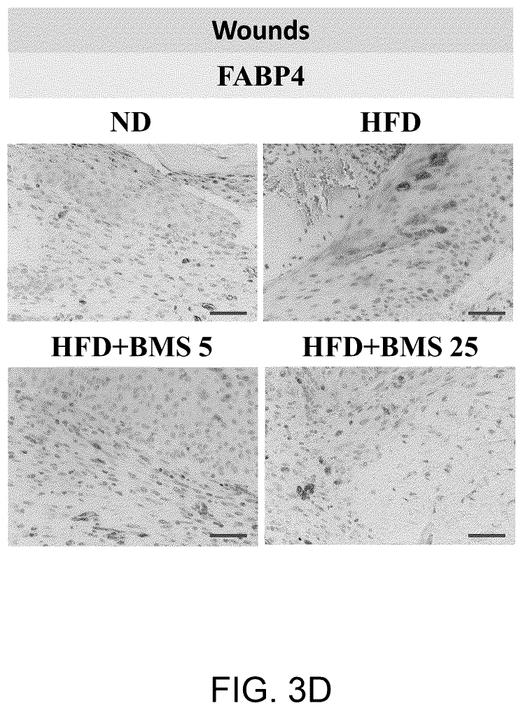

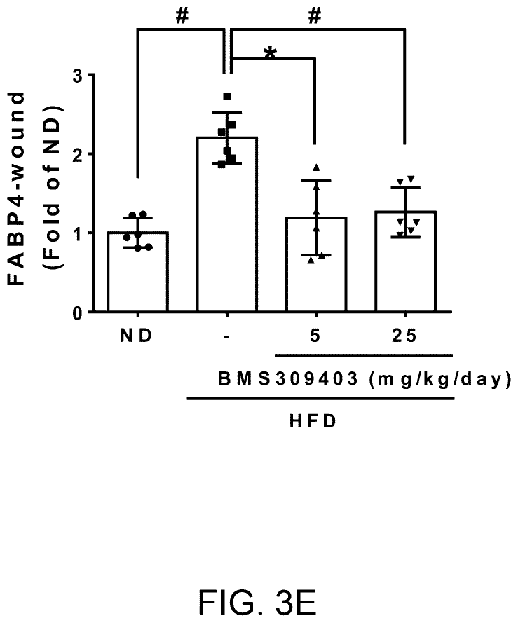

Continuing from Example 2, the mice were sacrificed on day 7 post-wounding, and wound tissues thereof were harvested for histological and immunostaining analysis. As shown in FIG. 3A, histological analysis of wound sections stained with H&E revealed that the untreated HFD mice exhibited a larger wound size relative to the ND mice. By contrast, the wound size of the BMS309403-treated HFD mice was smaller than that of the untreated HFD mice and the ND mice, indicating enhanced wound healing for the BMS309403-treated HFD mice. Furthermore, capillary density in the wound area was evaluated through the expression of CD31, a biomarker for capillaries and angiogenesis, by immunostaining. As shown in FIG. 3B and FIG. 3C, the untreated HFD mice exhibited significantly lower capillary density in the wound area compared to the ND mice. By contrast, BMS309403-treated mice exhibited significantly higher capillary density in the wound area compared to untreated metabolic disorder mice, indicating angiogenesis promotion during wound healing.

Moreover, immunostaining images in FIG. 3D, along with the quantitative analysis in FIG. 3E, confirms a significantly higher FABP4 expression at the wound area of the untreated HFD mice compared to the ND mice. By contrast, the wound area of BMS309403-treated HFD mice exhibited a significant reduction in FABP4 expression compared to untreated HFD mice.

These findings demonstrated that the administration of the FABP4 antagonist, BMS309403, suppresses the FABP4 expression in the wound area, thereby enhancing wound healing and promoting angiogenesis in the wound area. In addition, the in vivo wound healing assay of ND mice demonstrated that the BMS309403-treated ND mice exhibit higher CD31 expression in the wound area compared to untreated ND mice (data not shown), indicating inhibition of FABP4 promotes angiogenesis in not only chronic wounds but also acute wounds.

### Example 4: Restoration of Angiogenic Signaling in Wound Healing by FABP4 Inhibition

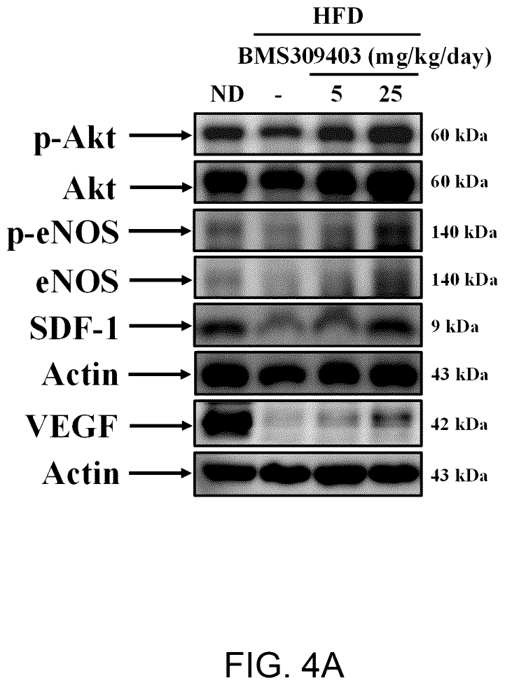

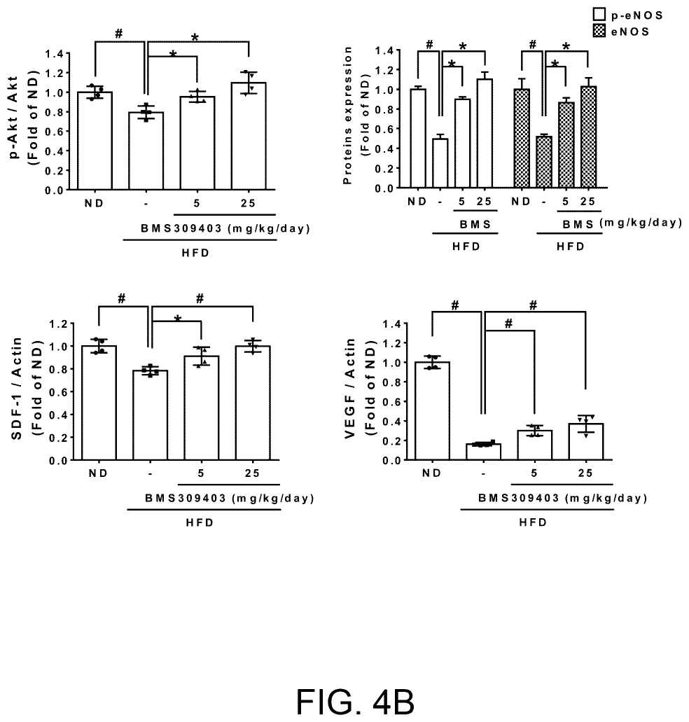

To investigate the effect of FABP4 inhibition on angiogenesis-associated signaling during wound healing of the HFD mice, wound tissues were harvested on day 7 post-wounding for protein expression analysis by western blot. Western blot images in FIG. 4A, along with the quantitative analysis in FIG. 4B, shows that, compared to the ND mice, the untreated HFD mice with metabolic disorders exhibited markedly reduced expression of key angiogenic factors, including phosphorylated AKT (p-AKT), phosphorylated endothelial nitric oxide synthase (p-eNOS), stromal cell-derived factor 1 (SDF-1), and VEGF in the wound area. These findings are indicative of compromised angiogenic signaling in the wound tissue of metabolically impaired mice. By contrast, the BMS309403-treated HFD mice showed significantly increased expression levels of p-AKT, p-eNOS, SDF-1, and VEGF when compared to the untreated HFD mice, which confirmed that FABP4 inhibition restored these angiogenic factors in the HFD mice to near those observed in the ND mice.

The elevation of p-AKT and p-eNOS suggests reactivation of the PI3K/AKT/eNOS signaling pathway, which is essential for endothelial cell activation, nitric oxide production, and vasodilation. Increased SDF-1 expression supports mobilization and recruitment of endothelial progenitor cells to the wound site. Upregulation of VEGF, a central regulator of angiogenesis, further indicates enhanced vascular remodeling and neovascularization.

Taken together, these results demonstrate that BMS309403 treatment effectively restores angiogenic signaling impaired by metabolic dysfunction and enhances the molecular processes involved in wound angiogenesis and tissue repair. In addition, the in vivo wound healing assay of ND mice demonstrated that the BMS309403-treated ND mice exhibit higher expression levels of p-AKT, p-eNOS, SDF-1, and VEGF in the wound area compared to untreated ND mice (data not shown), indicating inhibition of FABP4 restored these angiogenic factors in not only chronic wounds but also acute wounds.

### Example 5: Reduction of Inflammation and Scarring in Wound Healing by FABP4 Inhibition

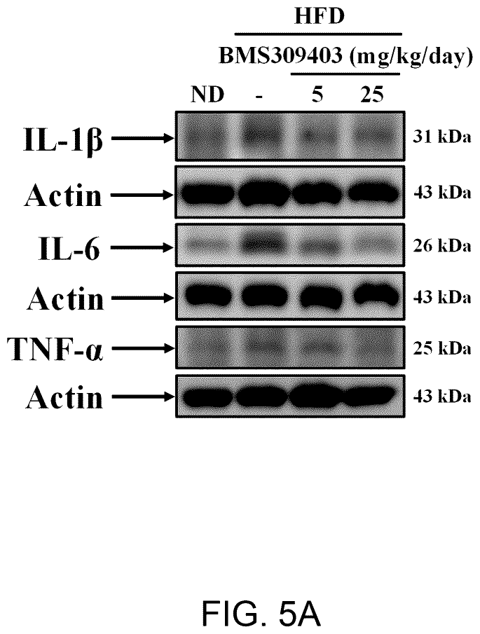

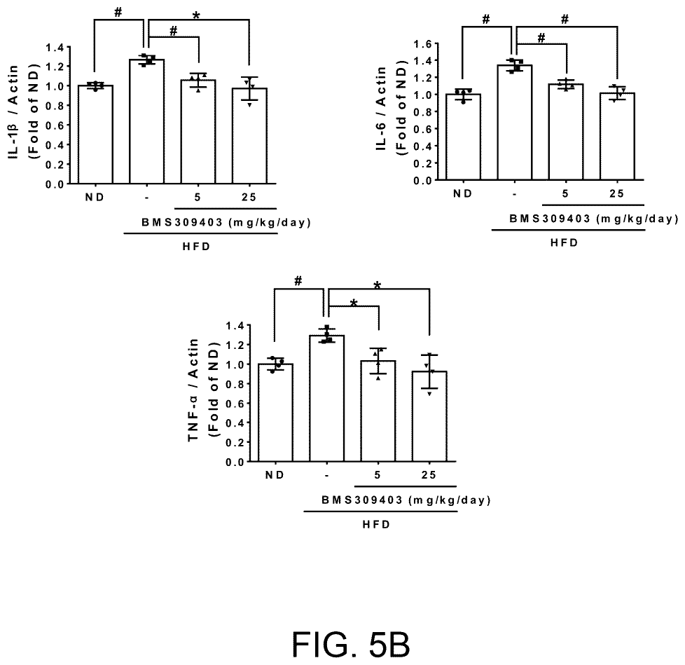

To investigate the effect of FABP4 inhibition on anti-inflammation during wound healing of the HFD mice, wound tissues were harvested on day 7 post-wounding for protein expression analysis by western blot. Western blot images in FIG. 5A, along with the quantitative analysis in FIG. 5B, shows that, compared to the ND mice, the untreated HFD mice with metabolic disorders exhibited markedly elevated expression of inflammatory factors including IL-1β, IL-6, and TNF-α in the wound area. These findings are indicative of increased or persistent inflammation in the wound tissue of metabolically impaired mice, which may also easily lead to scarring. By contrast, the BMS309403-treated HFD mice showed significantly decreased expression levels of IL-1β, IL-6, and TNF-α when compared to the untreated HFD mice, which confirmed that FABP4 inhibition reduced the wound inflammation in the HFD mice, thereby inhibiting the wound scarring. Also, no scar formation was observed in the BMS309403-treated HFD mice as compared with the untreated HFD mice. In addition, the in vivo wound healing assay of ND mice demonstrated that the BMS309403-treated ND mice exhibit lower expression levels of IL-1β, IL-6, and TNF-α in the wound area compared to untreated ND mice (data not shown), and no scar formation was observed in the BMS309403-treated ND mice as compared with the untreated ND mice. These data indicate that inhibition of FABP4 reduces wound inflammation and/or inhibits wound scarring in not only chronic wounds but also acute wounds.

In summary, the experiment demonstrated that the FABP4 antagonist promotes wound healing. The method utilizing the disclosed FABP4 antagonist can also promote angiogenesis, reduce inflammatory responses, and/or inhibit the wound scarring of the subject.

Those skilled in the art will readily observe that numerous modifications and alterations of the embodiments may be made while retaining the teachings of the present disclosure. Accordingly, the above disclosure should be construed as limited only by the metes and bounds of the appended claims.

What is claimed is:

1. A method for wound treatment, comprising administering a subject in need thereof with a pharmaceutical composition, wherein the pharmaceutical composition comprises:
an effective amount of a fatty acid-binding protein 4 antagonist and/or a pharmaceutically acceptable salt thereof; and
a pharmaceutically acceptable carrier.

2. The method of claim 1, wherein the fatty acid-binding protein 4 antagonist is at least one selected from the group consisting of fatty acid-binding protein 4 small molecule antagonist drugs, fatty acid-binding protein 4 neutralizing antibodies, and fatty acid-binding protein 4 gene interference ribonucleic acids.

3. The method of claim 1, wherein the fatty acid-binding protein 4 antagonist is BMS309403.

4. The method of claim 1, wherein the effective amount of the fatty acid-binding protein 4 antagonist is about 1 mg/kg to about 100 mg/kg.

5. The method of claim 4, wherein the effective amount of the fatty acid-binding protein 4 antagonist is about 5 mg/kg to about 25 mg/kg.

6. The method of claim 1, wherein a frequency of administration of the FABP4 antagonist or the pharmaceutical composition is three times a day, twice a day, once a day, once every two days, once every three days, once every four days, once every five days, once every six days, or once a week.

7. The method of claim 1, wherein the pharmaceutical composition is orally administered to the subject.

8. The method of claim 1, wherein the fatty acid-binding protein 4 antagonist and/or the pharmaceutically acceptable salt thereof is a sole active ingredient for the wound treatment of the subject.

9. The method of claim 1, wherein the wound is a chronic wound.

10. The method of claim 1, wherein the subject suffers from or is susceptible to a metabolic disease or disorder.

11. The method of claim 10, wherein the metabolic disease or disorder is at least one selected from the group consisting of diabetes, metabolic syndrome, and/or obesity.

12. The method of claim 1, wherein the pharmaceutical composition promotes wound angiogenesis of the subject.

13. The method of claim 12, wherein the pharmaceutical composition increases expression of a wound angiogenic factor comprising at least one selected from the group consisting of VEGF, SDF-1, EGF, Ang, EPO, FGF, GDF, eNOS, and AKT in the subject.

14. The method of claim 1, wherein the pharmaceutical composition enhances wound healing of the subject.

15. The method of claim 1, wherein the pharmaceutical composition reduces wound inflammation of the subject.

16. The method of claim 15, wherein the pharmaceutical composition decreases expression of a wound inflammatory factor comprising at least one selected from the group consisting of G-CSF, IL-1β, IL-1α, IL-2, IL-6, IL-8, IL-11, IL-17, IL-18, IFN-α, IFN-β, IFN-γ, TNF-α, and TNF-β in the subject.

17. The method of claim 1, wherein the pharmaceutical composition inhibits wound scarring of the subject.

18. The method of claim 1, wherein the pharmaceutical composition is orally administered to the subject to promote wound angiogenesis, enhance wound healing, reduce wound inflammation, and/or inhibit wound scarring of the subject, and the pharmaceutical composition comprises the effective amount of fatty acid-binding protein 4 antagonist and the pharmaceutically acceptable carrier.

19. The method of claim 18, wherein the fatty acid-binding protein 4 antagonist comprises BMS309403.

20. The method of claim 19, wherein the fatty acid-binding protein 4 antagonist is a sole active ingredient to promote the wound angiogenesis, enhance the wound healing, reduce the wound inflammation, and/or inhibit the wound scarring of the subject.
